(19) United States
(12) Patent Application Publication
(10) Pub. No.: US 2024/0238301 A1
(43) Pub. Date: Jul. 18, 2024

(54) COMPOSITIONS AND METHODS FOR REDUCED TOXICITY IN TRANSPLANTATION USING JANUS KINASE (JAK) INHIBITORS

(71) Applicant: THE REGENTS OF THE UNIVERSITY OF COLORADO, A BODY CORPORATE, Denver, CO (US)

(72) Inventors: Michael R. VERNERIS, Greenwood Village, CO (US); Laura Michelle MCLAUGHLIN, Denver, CO (US)

(21) Appl. No.: 18/622,525

(22) Filed: Mar. 29, 2024

Related U.S. Application Data
(63) Continuation of application No. PCT/US2022/045476, filed on Oct. 1, 2022.
(60) Provisional application No. 63/251,464, filed on Oct. 1, 2021.

(51) Int. Cl. A61K 31/519; A61K 35/28; A61P 37/06

(52) U.S. Cl. CPC A61K 31/519; A61K 35/28; A61P 37/06

(57) ABSTRACT

Embodiments of the instant disclosure generally relate to compositions and methods for improving engraftment, reducing the risk of cellular implantation or transplantation rejection, reducing toxicities due to radiation and chemotherapy treatments and/or treating a malignant or non-malignant condition or a subject having a solid organ transplantation using stem cell implantation in combination with compositions disclosed herein. In certain embodiments, compositions and methods disclosed herein concern administering a composition including, but not limited to, at least one Janus kinase (JAK) inhibitor. In some embodiments, compositions and methods disclosed herein can be administered in combination with and/or before, during and/or after hematopoietic cell transplantation (HCT) to treat a health condition in a subject.

### PRIORITY

This U.S. Continuation Application claims priority to International Application No. PCT/US2022/045476, filed Oct. 1, 2022, which claims priority under 35 U.S.C. § 119(e) to U.S. Provisional Application No. 63/251,464, filed Oct. 1, 2021. These applications are incorporated herein by reference in their entireties for all purposes.

### FIELD

Embodiments of the instant disclosure generally relate to compositions and methods for using one or more Janus kinase (JAK) inhibitor to improve outcome and success of hematopoietic cell transplantation (e.g., allogeneic HCT) and solid organ transplantation by reducing risk of transplant rejection, improving the longevity of the transplant (e.g., bone marrow or solid organ), improving immune cell recovery and/or treating a health condition in a subject.

### BACKGROUND

Allogeneic hematopoietic cell transplantation (allo-HCT) is a therapy for a variety of malignant and non-malignant conditions. In malignant conditions, this approach is used in the context of high dose chemotherapy and irradiation which can be used to treat the underlying malignancy, as well as impairing the recipient's immune system, facilitating engraftment of the transplanted donor stem cells. However, a growing percentage of pediatric patients (˜40% of all transplants at pediatric centers) receiving allo-HCT have non-malignant condition. In this context, the goal is not to treat the cancer, but rather to replace a defective hematopoietic system, immune system or to “cross-correct” a genetic disease by providing normal proteins (enzymes or otherwise). In these young patients, they do not have a malignancy; therefore, the treatment purpose is to use a reduced toxic approach to condition the patient, with the goal of impairing the immune system to permit engraftment. For these situations, a low dose chemotherapy alone or in combination with low dose total body irradiation is commonly used. It is well known that stem cell transplantation rejection is a common and life-threatening complication despite current treatment regimens. In addition, a significant number of older patients (>50 years old) are receiving reduced intensity transplantation due to ongoing medical issues that preclude tolerance to myeloablative conditioning with chemotherapy alone or chemotherapy and total body irradiation.

Solid organ transplantation, including kidney, liver, heart, lung, intestine and pancreas, is also used for the treatment of a variety of end-stage diseases. Following solid organ transplant, patients are maintained on lifelong immunosuppression in effort to prevent rejection of the transplanted organ. This chronic immunosuppression can put patients at high risk of developing other life-threatening complications, including, but not limited to malignancy. Even with continued immunosuppression, the median survival of a donated kidney, the most commonly transplanted organ, is around 10 years. Methods to overcome these immunological barriers to engraftment in both malignant and nonmalignant conditions are needed to improve outcome. Further, a need exists for immunosuppressive treatments to improve engraftment success with reduced side effects.

### SUMMARY

Embodiments of the instant disclosure relate to compositions and methods for using one or more Janus kinase (JAK) inhibitor alone or in combination with irradiation to improve hematopoietic cell transplantation (e.g., allogeneic HCT (allo-HCT), also known as bone marrow transplantation or stem cell transplantation) outcome for reducing the risk of transplant rejection, prolong transplantation of HCTs and/or treating a health condition. In certain embodiments, one or more JAK inhibitor can be combined with HCTs (or provided separately) and transplanted into a subject. In some embodiments, HCTs can include allogeneic HCTs (allo-HCTs) of use for transplantation. In certain embodiments, methods disclosed herein include administering HCTs and at least one JAK inhibitor before, during, simultaneously or after administering HCTs to a subject. In other embodiments, the subject has or is suspected of developing a condition in need of such a transplant.

Other embodiments disclosed herein relate to compositions and methods for using one or more Janus kinase (JAK) inhibitor alone or in combination with irradiation to improve solid organ transplant outcomes for reducing the risk of transplant rejection, prolong transplantation of solid organ or HCTs and/or treating a health condition. In certain embodiments, one or more JAK inhibitor can be combined with solid organ transplantation and transplanted into a subject. In some embodiments, methods disclosed herein include transplanting a solid organ along with at least one JAK inhibitor before, during, simultaneously or after solid organ transplantation of the subject. In certain embodiments, the subject has or is suspected of developing a condition in need of such a transplant. methods disclosed herein include transplanting a solid organ and treating the subject receiving such a transplant before and/or after transplantation with one or more JAK inhibitors. In some embodiments, these treatments with JAK inhibitors can be combined with conventional therapies to improve transplant acceptance and reduce or eliminate transplant rejection in the subject and treat the targeted health condition.

Compositions and methods known in the art used to improve engraftment longevity and reduce transplant rejection of HCTs as well as solid organ transplants rejection typically involve administration of toxic agents to inhibit a subject's immune system and reduce engraftment rejection. These toxic agents can cause long-term debilitating side effects. In subjects with non-malignant conditions, such as an infant, a child or young adult these side effects can have short and long-term effects due to using these toxic agents to improve engraftment and longevity of transplantation. In addition, older adults with malignant conditions and those who have undergone solid organ transplantation typically cannot tolerate myeloablative conditioning regimens associated with allo-HCT. There is a need for agents that are immune suppressive, short acting and have minimal short and/or long-term side effects for use in these subjects to improve outcome and reduce adverse effects of these agents. Embodiments disclosed herein have identified JAK inhibitors which are approved for use in other health conditions, such as myelofibrosis and autoimmune diseases (e.g., rheumatoid arthritis and ulcerative colitis) and have found that they improve HCT transplantation outcomes, for example, by preventing recipient immune responses against the donor and preventing rejection of the transplanted cells which lead to transplant tolerance (in the solid organ recipient) or amelioration of the disease or condition (malignant or non-malignant) in the transplant recipient. In certain embodiments, methods disclosed herein concern new uses for pre-existing agents alone or in combination with other treatments. In some embodiments, JAK inhibitor-containing compositions can be used in a pre-transplant conditioning phase to facilitate HCT engraftment for example, donor cell engraftment.

In some embodiments, methods disclosed herein can include administering at least one JAK inhibitor before infusing or introducing allo-HCT, after infusing or introducing allo-HCT, during infusion or introduction of allo-HCT, or any combination thereof. In accordance with these embodiments, the allo-HCT are maintained for prolonged periods or for the life-span of the subject compared to a subject not treated with JAK inhibitors. In other embodiments, methods disclosed herein include administering at least one JAK inhibitor-containing composition at least about 10 days or more, about 7 days, about 5 days, about 3 days, and/or about the day before and after infusing an allo-HCTs. In certain embodiments, methods disclosed herein include administering at least one JAK inhibitor-containing composition at least one of about 5 days before, at the time of, or within about a day or two days up to about 1 month and 2 months after administering an allo-HCT to the subject.

In some embodiments, a donor of stem cells or bone marrow can be pre-treated or simultaneously treated with a composition comprising one or more JAK inhibitor prior to, or at the time of harvesting donor stem cells or bone marrow. In certain embodiments, the subject can be treated with a composition comprising about 0.05 mg/kg to about 300 mg/kg JAK inhibitor as a single JAK inhibitor or a mixture of JAK inhibitors. In some embodiments, the subject is a human subject.

In certain embodiments, compositions and methods disclosed herein can include administering at least one JAK inhibitor-containing composition to a subject having an HCT transplant at a dose between about 0.05 mg/kg/day to about 300 mg/kg/day of the JAK inhibitor. In other embodiments, methods disclosed herein can include administering at least one of a JAK1 inhibitor, a JAK2 inhibitor, a JAK3 inhibitor, a JAK1/2 inhibitor, a JAK1/3 inhibitor, a JAK2/3 inhibitor, a JAK1/2/3 inhibitor, or any combination thereof. In certain embodiments, methods disclosed herein can include administering a chemotherapeutic agent combined with a JAK inhibitor, separately, simultaneously or after the one or more JAK inhibitor. In other embodiments, compositions including at least one JAK inhibitor can be used before, during or after chemotherapeutic agent treatments of a subject undergoing HCT in order to improve implantation, transplantation, or engraftment, reduce rejection of implanted blood, marrow and/or solid organ and/or reduce the concentration and/or frequency of chemotherapy and/or irradiation administered to the subject. In accordance with these embodiments, a JAK inhibitor can include, but are not limited to, ruxolitinib, tofacitinib, baricitinib, upadacitinib, fedratinib, peficitinib, decernotinib, filgotinib, solcitinib, itacitinib, SHR0302, abrocitinib, delgocitinib, cerdulatinib, gandotinib, lestaurtinib, momelotinib, pacritinib, deucravacitinib, cucurbitacin I, CHZ868, oclacitinib, XL019, AZD1480, BMS911543, ilginatinib, or any combination thereof.

In some embodiments, methods disclosed herein can further include exposing a subject having a malignant, a non-malignant condition or a condition requiring solid organ transplantation to irradiation in combination with administering a composition containing one or more JAK inhibitor. In certain embodiments, the subject can receive total body irradiation (TBI) of about 1 Gy to about 5 Gy before receiving HCT.

In some embodiments, the subject is a mammal. In certain embodiments, the mammal is a pet, livestock, or a horse. In other embodiments, the subject is a human subject. In yet other embodiments, the subject is a human subject of any age having a health condition in need of an allo-HCT or solid organ transplantation. In certain embodiments, the subject is an infant or a toddler or a child. In other embodiments, the subject is an adult such as an older adult.

In some embodiments, methods disclosed herein can include administering an allo-HCT and at least one JAK inhibitor to a subject having or suspected of developing a non-malignant or malignant condition. In accordance with these embodiments, the non-malignant condition can include, but is not limited to, bone marrow decline or failure, an immune dysregulation syndrome, a hemoglobinopathy, an immunodeficiency, a metabolic disorder, or other non-malignant condition in need of such a treatment or any combination thereof.

In some embodiments, methods disclosed herein can include administering HCTs (e.g. allo-HCT) and at least one JAK inhibitor to a subject having or suspected of developing a non-malignant disease, wherein the non-malignant disease can include, but is not limited to, familial hemophagocytic lymphohistiocytosis (FHL), Wiskott-Aldrich syndrome (WAS), T-cell deficiency, metachromatic leukodystrophy (MLD), Lagerhans cell histiocytocis, erythropoietic protoporphyria, adenosine deaminase deficiency (ADA), Maroteaux-Lamy syndrome (MPS VI), Fanconi syndrome, thrombocytopenia, aspartylglucosaminuria (AGU), adrenoleukodystrophy (ALD), chronic granulomatous disease (CGD), thalassemia major, Hurler syndrome, Kostmann syndrome, severe combined immunodeficiency (SCID), sickle cell anemia, paroxysmal nocturnal hemoglobinuria (PNH), beta thalassemia, aplastic anemia or any disease that can be be treated with allogeneic transplantation or any combination thereof. In some embodiments, methods disclosed herein include, but are not limited to, providing an HCT (e.g., allo-HCT) and at least one JAK inhibitor to a subject, where the subject having or suspected of having a non-malignant disease does not have or is not suspected of having myelofibrosis. In other embodiments, a subject having a malignant condition contemplated herein can include leukemia or other malignant condition, and at least one JAK inhibitor can be used before, during or after transplantation such as HCT in order improve implant outcome while reducing the need for chemotherapeutic agents and/or irradiation. In some embodiments, compositions containing at least one JAK inhibitor can be used to ameliorate the side effects, reduce, or eliminate chemotherapy and/or radiation exposure in any subject receiving HCT for any health condition. In certain embodiments, the health condition can include, but not limited to, administration of HCT to induce graft tolerance, and subsequently reduce or eliminate the need for short-term and even lifelong immunosuppression following solid organ transplantation.

In other embodiments, compositions for reducing cellular implantation rejection in a subject are contemplated herein. In some embodiments, compositions for reducing cellular implantation rejection in a subject following HCT (e.g. allo-HCT) can include at least one JAK inhibitor and at least one pharmaceutically acceptable excipient. In certain embodiments, compositions disclosed herein including at least one JAK inhibitor can include, but are not limited to, ruxolitinib, tofacitinib, baricitinib, upadacitinib, fedratinib, peficitinib, decernotinib, filgotinib, solcitinib, itacitinib, SHR0302, abrocitinib, delgocitinib, cerdulatinib, gandotinib, lestaurtinib, momelotinib, pacritinib, deucravacitinib, cucurbitacin I, CHZ868, oclacitinib, XL019, AZD1480, BMS911543, ilginatinib, or other JAK inhibitor, or any combination thereof. In other embodiments, compositions disclosed herein can include about 1 mg to about 300 mg of at least one JAK inhibitor.

In some embodiments, compositions disclosed herein can be formulated for intravenous administration. In other embodiments, compositions disclosed herein can be formulated for oral administration. In certain embodiments, compositions disclosed herein can be formulated for human use (e.g., pediatric use) or other mammals.

In some embodiments, the present disclosure provides methods of treating a non-malignant or malignant condition being treated by HCT to improve engraftment and reduce side effects of other standard treatments. In other embodiments, the present disclosure provides methods of administering HCT to subject preparing for or scheduled to receive or have received a solid organ transplant for inducing tolerance to the grafted organ, to reduce or eliminate the need for immunosuppression including short-term or lifelong immunosuppression. In accordance with these embodiments, methods of treating these conditions can include allo-HCT infusion separately, or in combination with compositions including at least one JAK inhibitor to a subject, where the subject has or is suspected of developing a non-malignant condition. In certain embodiments, the subject does not have or is not suspected of developing myelofibrosis.

In some embodiments, the present disclosure provides kits for preparing, storing, and/or administering compositions disclosed herein. In certain embodiments, the present disclosure provides kits for practicing any of the methods disclosed herein.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIG. 1 represents an exemplary experiment illustrating the generation and assessment of allogeneic bone marrow transplant mouse models using an experimental mouse model as recipients and other mice as donors in accordance with certain embodiments of the present disclosure.

FIG. 2 represents an exemplary experiment illustrating transplant outcomes of either successful engraftment of donor cells or rejection of donor cells present in mouse models treated with and without JAK inhibitors for a predetermined period after allogeneic bone marrow transplant using certain mice as recipients and another mouse strain as donors in accordance with certain embodiments of the present disclosure.

FIG. 3 represents an exemplary experiment illustrating the generation and assessment of allogeneic bone marrow transplant mouse models using certain mice as recipients and another mouse strain as donors in accordance with certain embodiments of the present disclosure.

FIG. 4 represents an exemplary experiment illustrating the transplant outcomes of either successful engraftment of donor cells or rejection of donor cells in mouse models treated with and without JAK inhibitors for a predetermined after allogeneic bone marrow transplant using one mouse strain as recipients and another mouse strain as donors in accordance with certain embodiments of the present disclosure.

FIG. 5 represents an exemplary experiment illustrating percent donor engraftment in mouse models treated with JAK inhibitors for predetermined period assaying at various times after allogeneic bone marrow transplant using a mouse model as recipients and another mouse strain as donors in accordance with certain embodiments of the present disclosure.

FIG. 6 represents an exemplary experiment illustrating methods for assessing JAK inhibitor effects on recipient and donor T cells in allogeneic bone marrow transplant mouse models in accordance with certain embodiments of the present disclosure.

FIGS. 7A-7C represent exemplary experiments illustrating effects in the presence and absence of a chemotherapeutic agent effects on the number of total recipient NK+ cells (FIG. 7A) CD3+ T cells (FIG. 7B); CD4+ T cells (FIG. 7C); and CD8+ T cells (FIG. 7D) in accordance with certain embodiments of the present disclosure.

FIG. 8 represents an exemplary experiment illustrating the generation and assessment of allogeneic bone marrow transplant mouse models using one mouse strain as recipients and another mouse strain as donors in accordance with certain embodiments of the present disclosure.

FIG. 9 represents an exemplary experiment illustrating percent donor engraftment in mouse models treated with JAK inhibitors over several weeks after allogeneic bone marrow transplant using one mouse strain as recipients and another mouse strain as donors in accordance with certain embodiments of the present disclosure.

FIGS. 10A-10D represent exemplary experiments illustrating examination of T cells several weeks after allogeneic bone marrow transplantation (FIG. 10A) overall T cell percentages in the various conditions; (FIG. 10B) CD4+ and CD8+ T cell percentages; (FIG. 10C) Percentage of donor CD4+ T cells; (FIG. 10D) percentage of donor CD8+ T cells in accordance with certain embodiments of the present disclosure.

FIG. 11 represents an exemplary experiment illustrating examination of hematopoietic stem cells (HSCs) several weeks after allogeneic bone marrow transplantation demonstrating engraftment of long-term engrafting stem cells (Lin−LSK+CD48−CD150+) in accordance with certain embodiments of the present disclosure.

### DEFINITIONS

Terms, unless defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

Unless otherwise defined, all technical terms used herein have the same meaning as commonly understood by one of ordinary skill in the art to which this disclosure belongs.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the instant disclosure relate to novel compositions, methods, and kits including compositions such as pharmaceutical compositions containing at least one Janus kinase (JAK) inhibitor for administering to a subject scheduled to undergo or undergoing HCT transplantation. In certain embodiments, the at least one JAK inhibitor can be administered to the subject in conjunction with allo-HCT for example, before, during or after allo-HCT infusion or administration to the subject. In accordance with these embodiments, the at least one JAK inhibitor reduces HCT rejection, improves HCT engraftment, prolongs HCT transplantation in the subject and can further reduce or eliminate the need for irradiation and/or a chemotherapeutic agents to inhibit the immune system while reducing toxic side effects of these treatment in the subject. In other embodiments, allo-HCTs can be administered before, during or after solid organ transplantation. In accordance with these embodiments, at least one JAK inhibitor can be used to reduce or eliminate HCT rejection, improve HCT engraftment, and/or prolong HCT transplantation in the subject to reduce or eliminate the need for continued immunosuppression as well as to reduce or prevent rejection following solid organ transplantation.

In some embodiments, compositions disclosed herein can include, but are not limited to, at least one JAK inhibitor. Janus kinases (JAK) are a family four enzymes; JAK1, JAK2, JAK3 and tyrosine kinase 2 (TYK2) that are proximal signaling molecules downstream of most cytokine and growth factor receptors. In some embodiments, JAK inhibitors can be small molecules, peptides, antibodies, and the like that competitively inhibit signaling through these receptors or act on the kinases directly. In certain embodiments, JAK inhibitors can have broad biodistribution, a short serum half-life, minimal to no long-term side effects, or any combination thereof. In other embodiments, JAK inhibitors can be specific inhibitors of JAK1. Non-limiting examples of JAK1 inhibitors for use herein alone or in combination with other JAK inhibitors include, but are not limited to, nacitinib, upadacitinib, filgotinib, G-146034, solcitinib, itacitinib, abrocitinib, or a combination thereof. In some embodiments, JAK inhibitors can be specific inhibitors of JAK2. Non-limiting examples of JAK2 inhibitors for use herein alone or in combination with other JAK inhibitors include, but are not limited to, fedratinib, gandotinib, lestaurtinib, pacritinib, CHZ868 or a combination thereof. In certain embodiments, JAK inhibitors can be a specific inhibitor of JAK3. Non-limiting examples of JAK3 inhibitors for use herein alone, or in combination with other JAK inhibitors include, but are not limited to, tofacitinib, peficitinib, decernotinib or a combination thereof. In some embodiments, JAK inhibitors can be a dual inhibitor of two members of the JAK family. In accordance with these embodiments, a dual JAK inhibitor can include a JAK1/JAK2 inhibitor. Non-limiting examples of dual JAK1/JAK2 inhibitors for use in compositions disclosed herein include, but are not limited to, ruxolitinib, baricitinib, momelotinib, CTP-543, or a combination thereof. In accordance with these embodiments, a dual JAK inhibitor can include a TYK2/JAK inhibitor. Non-limiting examples of dual TYK/JAK inhibitors for use herein can include cerdulatinib, PF-06700841 or a combination thereof. In other embodiments, a dual JAK inhibitor can be a JAK2/JAK3 inhibitor. In some embodiments, a dual JAK inhibitor can be a JAK1/JAK3 inhibitor. In other embodiments, JAK inhibitors of use to reduce HCT implantation rejection and prolong engraftment can include JAK inhibitors that inhibit all members of the JAK family (e.g., a pan-JAK inhibitor). A non-limiting example of a pan-JAK inhibitor for use in compositions and methods disclosed herein can include, but is not limited to, peficitinib.

In some embodiments, compositions disclosed herein can include at least one JAK inhibitor including, but not limited to, ruxolitinib, tofacitinib, baricitinib, upadacitinib, fedratinib, peficitinib, decernotinib, filgotinib, solcitinib, itacitinib, SHR0302, abrocitinib, delgocitinib, cerdulatinib, gandotinib, lestaurtinib, momelotinib, pacritinib, deucravacitinib, cucurbitacin I, CHZ868, oclacitinib, XL019, AZD1480, BMS911543, ilginatinib, or any combination thereof alone or in combination with cells to be implanted or infused. In certain embodiments, compositions disclosed herein can include, but are not limited to, ruxolitinib, tofacitinib, or any combination thereof alone or in combination with cells to be implanted or infused.

In some embodiments, compositions disclosed herein can include about 1.0 mg to about 400 mg; about 0.05 mg to about 350 mg; about 0.05 to about 250 mg; about 0.05 to about 200 mg; about 0.05 to about 150 mg; about 0.05 to about 125 mg, about 0.05 mg to about 100 mg, about 0.05 to about 75; about 0.05 to about 50 mg, about 0.05 to about 25 mg) of at least one JAK inhibitor. In certain embodiments, compositions disclosed herein can include about 0.05 mg to about 400 mg (e.g., about 0.05 mg, about 1 mg, 5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg or about 400 mg) total concentration of one or more JAK inhibitor(s) in a formulation.

In some embodiments, at least one JAK inhibitor treatment regimen can include a regimen beginning about 6 months or about 5 months or about 4 months or less before implantation (e.g., HCT) to about 1 day, to about 3 days, to about a week, to about 10 days, to about 2 weeks, to about 1 month, to about 2 months to up to about 6 months after implantation. In certain embodiments, JAK inhibitors can be given to a subject undergoing HCT once daily, twice daily, every other day, every few days or as needed to the subject. In other embodiments, at least one JAK inhibitor can be provided to a subject undergoing HCT by administering the composition two times daily at dosing levels contemplated herein; for example, one dose administered in the morning and then another dose administered in the late afternoon to evening.

In certain embodiments, pharmaceutically compositions are contemplated. In accordance with these embodiments, pharmaceutical compositions can include one or more JAK inhibitors disclosed herein and at least one pharmaceutically acceptable diluent(s), excipient(s), and/or carrier(s). As used herein, the term “pharmaceutically acceptable” refers to those compounds, materials, compositions, and/or dosage forms which are, within the scope of sound medical judgment, suitable for use in contact with the tissues, organs, and/or bodily fluids of a subject without excessive toxicity, irritation, allergic response, or other problems or complications commensurate with a reasonable benefit/risk ratio. Pharmaceutically acceptable diluents, carriers, and excipients for use disclosed herein can include, but are not limited to, physiological saline, Ringer's solution, phosphate solution or buffer, buffered saline, and other carriers known in the art. In some embodiments, compositions disclosed herein can also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof.

As used herein, the term “pharmaceutically acceptable excipient” can refer to solvents, dispersion media, coatings, antibacterial agents, antifungal agents, isotonic and absorption delaying agents, or the like that are physiologically compatible. Pharmaceutically acceptable excipients suitable for use herein, include, but are not limited to, buffers that are well known in the art, and can be phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

In some embodiments, pharmaceutical compositions herein can be formulated in a conventional manner using one or more physiologically acceptable carriers, excipients and/or auxiliaries. In some embodiments, pharmaceutical compositions disclosed herein can be an aqueous suspension having one or more polymers as suspending agents. In some embodiments, polymers for use in compositions disclosed herein can include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some embodiments, pharmaceutical compositions disclosed herein can include at least 5%, at least 10%, at least 20%, at least 25%, at least 30%, at least 35%, at least 40%, at least 45%, or at least 50% total amount of polymers as suspending agent(s) by total weight of the composition.

In some embodiments, pharmaceutical compositions disclosed herein can be a viscous formulation. In certain embodiments, viscosity of a pharmaceutical formulation can be increased by the addition of one or more gelling or thickening agents. In some embodiments, pharmaceutical compositions disclosed herein can have at least 5%, at least 10%, at least 20%, at least 25%, at least 30%, at least 35%, at least 40%, at least 45%, or at least 50% total amount of gelling or thickening agent(s) by total weight of the composition. In some embodiments, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In some embodiments, viscosity enhancing agents can be acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof.

In some embodiments, pharmaceutical compositions can include additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In other embodiments, pharmaceutical compositions disclosed herein can have at least 5%, at least 10%, at least 20%, at least 25%, at least 30%, at least 35%, at least 40%, at least 45%, or at least 50% total amount of one or more agents by total weight of the composition. In some embodiments, pharmaceutical compositions herein can have one or more of these agents added to improve the performance, efficacy, safety, shelf-life and/or other property of the composition or active agent (e.g., JAK inhibitor) of the present disclosure. In certain embodiments, additives can be biocompatible, and not be harsh, abrasive, or allergenic.

In other embodiments, pharmaceutical compositions disclosed herein can include one or more acidifying agents. As used herein, “acidifying agents” refers to compounds used to provide an acidic medium. Such compounds can include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid can be used. In certain embodiments, pharmaceutical compositions disclosed herein can have at least 5%, at least 10%, at least 20%, at least 25%, at least 30%, at least 35%, at least 40%, at least 45%, or at least 50% total amount of one or more acidifying agents by total weight of the composition.

In some embodiments, pharmaceutical compositions can include one or more alkalizing agents. As used herein, “alkalizing agents” are compounds used to provide alkaline medium. Such compounds can include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In other embodiments, any pharmaceutically acceptable organic or inorganic base can be used. In some embodiments, pharmaceutical compositions disclosed herein can have at least 5%, at least 10%, at least 20%, at least 25%, at least 30%, at least 35%, at least 40%, at least 45%, or at least 50% total amount of one or more alkalizing agents by total weight of the composition.

In other embodiments, pharmaceutical compositions herein can include one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some embodiments, pharmaceutical compositions disclosed herein can have at least 5%, at least 10%, at least 20%, at least 25%, at least 30%, at least 35%, at least 40%, at least 45%, or at least 50% total amount of one or more antioxidants by total weight of the composition.

In some embodiments, pharmaceutical compositions can include a buffer system. As used herein, a “buffer system” includes one or more buffering agents wherein “buffering agents” can be compounds used to resist change in pH upon dilution or addition of acid or alkali or maintain the pH of the composition. Buffering agents can include, by way of example and without limitation, potassium metaphosphate, potassium phosphate, monobasic sodium acetate and sodium citrate anhydrous and dihydrate and other agents known to one of ordinary skill in the art. In some embodiments, any pharmaceutically acceptable organic or inorganic buffer can be used. In some embodiments, pharmaceutical compositions disclosed herein can have at least 5%, at least 10%, at least 20%, at least 25%, at least 30%, at least 35%, at least 40%, at least 45%, or at least 50% total amount of one or more buffering agents by total weight of the composition. In some embodiments, the amount of one or more buffering agents can depend on the desired pH of the composition herein. In other embodiments, pharmaceutical compositions disclosed herein can have a pH of about 3 to about 9 (e.g., about 3. 4. 5. 6. 7. 8. 9). In certain embodiments, the pH of the composition when combined with HCT for implantation will be maintained at a pH suitable to preserve the cells and for implantation such as a physiological pH or range of pH.

In some embodiments, pharmaceutical compositions disclosed herein can include one or more preservatives. As used herein, “preservatives” refers to agents or combination of agents that inhibits, reduces or eliminates bacterial growth in a pharmaceutical dosage form. Non-limiting examples of preservatives can include Nipagin, Nipasol, isopropyl alcohol, methyl or propyl-p-hydroxybenzoates, sorbic acid, and a combination thereof. In some embodiments, any pharmaceutically acceptable preservative can be used. In other embodiments, pharmaceutical compositions disclosed herein can have at least 5%, at least 10%, at least 20%, at least 25%, at least 30%, at least 35%, at least 40%, at least 45%, or at least 50% total amount of one or more preservatives by total weight of the composition.

In some embodiments, pharmaceutical compositions disclosed herein can include one or more surface-acting reagents or detergents. In some embodiments, surface-acting reagents or detergents can be synthetic, natural, or semi-synthetic. In other embodiments, pharmaceutical compositions disclosed herein can include anionic detergents, cationic detergents, zwitterionic detergents, ampholytic detergents, amphoteric detergents, nonionic detergents having a steroid skeleton, or a combination thereof. In some embodiments, pharmaceutical compositions disclosed herein can include at least 5%, at least 10%, at least 20%, at least 25%, at least 30%, at least 35%, at least 40%, at least 45%, or at least 50% total amount of one or more surface-acting reagents or detergents by total weight of the composition.

In some embodiments, pharmaceutical compositions disclosed herein can include one or more stabilizers. As used herein, a “stabilizer” refers to a compound used to stabilize an active agent against physical, chemical, or biochemical process that would otherwise reduce the therapeutic activity of the agent. Suitable stabilizers can include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some embodiments, pharmaceutical compositions disclosed herein can include at least 5%, at least 10%, at least 20%, at least 25%, at least 30%, at least 35%, at least 40%, at least 45%, or at least 50% total amount of one or more stabilizers by total weight of the composition.

In other embodiments, pharmaceutical compositions disclosed herein can include one or more tonicity agents. As used herein, a “tonicity agents” refers to a compound that can be used to adjust the tonicity of the liquid formulation. Suitable tonicity agents can include, but are not limited to, glycerin, lactose, mannitol, dextrose, sodium chloride, sodium sulfate, sorbitol, trehalose and others known to those or ordinary skill in the art. In some embodiments, pharmaceutical compositions herein can include at least 5%, at least 10%, at least 20%, at least 25%, at least 30%, at least 35%, at least 40%, at least 45%, or at least 50% total amount of one or more tonicity modifiers by total weight of the composition.

In certain embodiments, pharmaceutical compositions disclosed herein can be formulated for one or more routes of administration. In some embodiments, routes of administration contemplated for pharmaceutical compositions herein can include, but are not limited to, oral, rectal, transmucosal, or parenteral delivery, including intramuscular, subcutaneous and intramedullary injections as well as, intravenous, by infusion such as renal vein infusion, intraperitoneal, intranasal injections. In some embodiments, pharmaceutical compositions disclosed herein can be formulated for local and/or systemic administration. In some embodiments, pharmaceutical compositions disclosed herein can be administered parenterally, e.g., by intravenous injection, intracerebroventricular injection, intra-cisterna magna injection, intra-parenchymal injection, venous infusion, or a combination thereof. In other embodiments, pharmaceutical compositions disclosed herein can be administered orally.

In certain embodiments, pharmaceutical compositions disclosed herein can be prepared by processes well known in the art, e.g., by means of conventional mixing, dissolving, granulating, levigating, emulsifying, encapsulating, entrapping, or lyophilizing processes. In other embodiments, pharmaceutical compositions for use in accordance with the present disclosure can be formulated in conventional manner using one or more physiologically acceptable carriers, excipients, and/or auxiliaries, which facilitate processing of the active ingredients (e.g., JAK inhibitors) into preparations which, can be used pharmaceutically. In certain embodiments, formulation of the pharmaceutical compositions herein can be dependent upon the route of administration chosen. In some embodiments, the active agents (e.g., JAK inhibitors) can be formulated in aqueous solutions for injection, where aqueous solutions can be physiologically compatible buffers such as Hank's solution, Ringer's solution, or physiological salt buffer.

In certain embodiments, pharmaceutical compositions disclosed herein can be formulated for parenteral administration, e.g., by bolus injection or intermittent or continuous infusion. In some embodiments, pharmaceutical compositions disclosed herein can be suspensions, solutions, or emulsions in oily or aqueous vehicles, and can contain agents such as suspending, stabilizing and/or dispersing agents. In some embodiments, pharmaceutical compositions disclosed herein can be formulated for oral, buccal or sublingual administration, such as in the form of tablets, capsules, ovules, elixirs, solutions or suspensions, which can contain flavoring or coloring agents, for immediate-, delayed- or controlled-release applications. In some embodiments, a donor can be pre-treated or simultaneously administered a composition containing one or more JAK inhibitor while donating stem or bone marrow cells for use in a subject in need thereof. It is contemplated that the transfer or HCT infusion can occur at the time of or shortly after removal of the HCs from a donor subject where at least one of the donor and/or recipient receives a JAK inhibitor-containing formulation.

In some embodiments, pharmaceutical compositions can be in the form of tablets. In accordance with some embodiments herein, tablets contemplated herein can contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. In accordance with some embodiments disclosed herein, tablets contemplated herein can contain lubricating agents such as magnesium stearate, stearic acid, glyceryl behenate and talc. In other embodiments, pharmaceutical compositions herein can be solid compositions employed as fillers in gelatin capsules. In certain embodiments herein, excipients included in gelatin capsules contemplated herein can include lactose, starch, cellulose, milk sugar or high molecular weight polyethylene glycols.

In some embodiments, pharmaceutical compositions herein can be presented in unit-dose or multi-dose containers, for example sealed ampoules or vials, and can be stored in a freeze-dried (lyophilized) condition requiring only the addition of the sterile liquid carrier immediately prior to use. In other embodiments, pharmaceutical compositions herein can be formulated for pediatric use. Children (e.g., 0-17 years) can require different oral dosage forms from adults due to differences in swallowing abilities, taste preferences, and dosage requirements. In certain embodiments, pharmaceutical compositions disclosed herein can be formulated for pediatric use, e.g., for special or preferred applicability to children. Non limiting examples of compositions formulated for children can include lozenges, dissolvable wafers or dissolvable patches, lollies (e.g., lollypops, “pops” or suckers), candies, gums, aerosols, sprays, powders, and the like. In other embodiments, pharmaceutical compositions disclosed herein can be formulated for pediatric use wherein the composition can be reconstituted in food or drink prior to oral administration or other form suitable for a young child.

In certain embodiments, the present disclosure provides methods for treating a non-malignant disease in a subject in need thereof. In some embodiments, the present disclosure provides methods of administering allo-HCT and at least one JAK inhibitor as disclosed herein to a subject, where the subject has or is suspected of having a non-malignant disease. In other embodiments, to practice the methods described herein, an effective amount of a JAK inhibitor or a pharmaceutical composition including a JAK inhibitor as disclosed herein can be administered to a subject who needs treatment via a suitable route (e.g., intravenous infusion or oral administration of a JAK inhibitor).

In certain embodiments, a subject to be treated by any of the methods disclosed herein can be a mammal (e.g., a human patient or a non-human primate). In some embodiments, a subject to any of the methods disclosed herein can have, be suspected of having, or be at risk for a non-malignant condition. In accordance these embodiments, a non-malignant condition suitable for treatment by any method disclosed herein can include, but is not limited to, bone marrow failure, immune dysregulation syndrome, hemoglobinopathy, immunodeficiency, metabolic disorder, or any combination thereof. In some embodiments, a non-malignant condition suitable for treatment by any method disclosed herein can include a genetic disorder. In other embodiments, a non-malignant disease suitable for treatment by any method disclosed herein can include, but is not limited to, familial hemophagocytic lymphohistiocytosis (FHL), Wiskott-Aldrich syndrome (WAS), T-cell deficiency, metachromatic leukodystrophy (MLD), Langerhans cell histiocytosis, erythropoietic protoporphyria, adenosine deaminase deficiency (ADA), Maroteaux-Lamy syndrome (MPS VI), Fanconi syndrome, thrombocytopenia, aspartylglucosaminuria (AGU), adrenoleukodystrophy (ALD), chronic granulomatous disease (CGD), thalassemia major, Hurler syndrome, Kostmann syndrome, severe combined immunodeficiency (SCID), sickle cell anemia, paroxysmal nocturnal hematoblobunimeuria (PNH), severe aplastic anemia (SAA), immune dysregulation syndromes such as X-linked lympoproliferation (XLP) and others, STAT-3 gain-of-function or a combination thereof. In other embodiments a health condition to be treated can include autoimmune conditions such as Crohn's disease, multiple sclerosis (MS), lupus, Behcet's or a combination thereof. In other embodiments, the subject does not have an autoimmune disorder such as rheumatoid arthritis. In certain embodiments, a subject to be treated by compositions and methods disclosed herein having a non-malignant disease can be identified by routine medical examination, e.g., laboratory tests, genetic tests, metabolic tests, organ functional tests, CT scans, and/or ultrasounds. In other embodiments, a subject to be treated by compositions and methods disclosed herein having a non-malignant condition does not have myelofibrosis or an autoimmune condition such as rheumatoid arthritis. In yet other embodiments, a subject having a malignant condition can include, but is not limited to, ALL, AML, lymphoma, MDS, myeloma, CLL and any other malignant condition treated with stem cell or HCT infusion.

In certain embodiments, a subject contemplated herein is in need of solid organ transplantation. In accordance with these embodiments, the subject can have a disease, a genetic condition or other condition requiring solid organ transplantation. In certain embodiments, the subject is experiencing organ failure such as liver, heart, kidney, or other organ failure. In some embodiments, a solid organ can include the pancreas or a component of the gastrointestinal (GI) tract. In other embodiments, a solid organ can include the liver, heart, kidney, or other solid organ to be replaced in the subject. In accordance with these embodiments, the subject can be treated with allo-HCT before, at the time of, or after receiving a replacement organ or organs and treated with one or more JAK inhibitors disclosed herein to enhance engraftment outcome. In yet other embodiments, the subject is undergoing or has undergone a cellular transplantation such as pancreatic islet cell transplantation. In yet other embodiments, the subject has suffered from burns and is undergoing skin grafts where an allo-HCT can improve outcome by increasing skin graft acceptance by the subject. In accordance with these embodiments, one or more JAK inhibitor can be provided to the subject before, during or after transplantation as described herein to reduce the need for toxic immunosuppressive agents and/or irradiation and improve engraftment.

In some embodiments, a subject to be treated by methods disclosed herein can be a human subject or other subject such as a non-human mammalian subject (a pet, livestock, horse, or other animal). In other embodiments, a subject to be treated by methods disclosed herein can be a human subject 20 years of age or older. In certain embodiments, a subject to be treated by compositions and methods disclosed herein can be a human subject. In certain embodiments, a subject to be treated by compositions and methods disclosed herein can be less than 20 years of age, such as a young adult, a teenager, a child, an infant or a fetus. In further embodiments, a subject can have a condition where the subject is in need or has had a solid organ transplant including, but not limited to, kidney, liver, lung, heart, pancreas and intestine transplant whom can undergo allo-HCT prior to, at the same time, or after the solid organ transplantation in order to enhance or establish a subject's immune tolerance to the transplanted solid organ. In accordance with these embodiments, such a subject undergoing HCT prior to, at the same time, or after the solid organ transplantation can lead to a reduction of exposure to, or reduce the duration of immunosuppression to prevent organ rejection using one or more immunosuppressive agent.

In other embodiments, a subject to be treated by methods disclosed herein can be a human child subject. In certain embodiments, a human child subject can be younger than 18 years (e.g., 0-17 years of age). In other embodiments, a human subject to be treated by methods disclosed herein can be younger than 12 years old (e.g., about 1, 2, 3, 4, 5, 6, 7, 8, 9, 20, 11). In certain embodiments, a human subject to be treated by compositions and methods disclosed herein can be an infant, e.g., younger than 12 months. In some embodiments, a human subject to be treated by methods disclosed herein can be an adolescent (e.g., 16-20 years old) or an adult.

In some embodiments, compositions and methods disclosed herein can be used for reducing graph rejection or implantation rejection in a subject following HCT (e.g. allo-HCT). In certain embodiments, administering a composition disclosed herein having one or more JAK inhibitors to a subject in need thereof scheduled to, or undergoing HCT as disclosed herein can reduce implantation rejection in the subject by about 1% to about 100% (e.g., about 1%, 5%, 10%, 20%, 30%, 40%, 50%, 60%, 70%, 80%, 90%, 95%, 99%, 100%) compared to a subject scheduled to or undergoing HCT (e.g. allo-HCT) not administered a composition including JAK inhibitors.

In some embodiments, compositions and methods disclosed herein can be used for reducing graft rejection in a subject following allo-HCT by reducing or eliminating allo- and/or anti-donor antibody production and/or T-cell and NK cell responses in organs and in tissues in a subject preparing for, undergoing, or having undergone allo-HCT. In certain embodiments, compositions and methods disclosed herein concern inducing immune tolerance by reducing or eliminating allo-antibody production and/or responses to allografts received from a donor. In accordance with these embodiments, the donor can be a major histocompatibility complex (MHC) fully matched, a partial MHC matched (e.g., 1-5 MHC alleles [HLA-A, -B, -C, -DRB1, -DQ or -DP] or even haplo-mismatched) or a fully MHC mismatched donor compared to the recipient receiving the transplant where compositions and methods disclosed herein can reduce or eliminate allo-antibody production. In some embodiments, the subject can be a human subject and compositions and methods disclosed herein can be used to induce transplant or infusion tolerance from a donor as an MHC matched (or HLA matched), a partial MHC matched or a fully mismatched donor such as a human donor compared to the human recipient receiving allo-HCT. In other embodiments, the compositions and methods disclosed herein can be used for solid organ transplantation from MHC matched, partially or fully matched donors. In accordance with these embodiments, to reduce or eliminate allo-antibody production in allo-graft transplantation, a subject scheduled for, or having an allograft transplantation from a donor, the subject can be administered a composition herein that includes at least one JAK inhibitor.

In some embodiments, methods herein can improve engraftment, prolong graft survival, reduce implant rejection and improve outcome of the subject receiving such an allo-HCT by inducing engraftment and/or tolerance and/or reducing and/or eliminating antibody-mediated response (AMR) to the donor implant or graft and/or modulate T- or NK-cell mediated immunities related to graft rejection and/or rejections by the graft (e.g. GvHD).

In certain embodiments, T or NK-cell production in organs and/or tissues can be functionally inhibited, blocked, or transiently depleted following administration of compositions herein. In some embodiments, compositions and methods disclosed herein can be used to suppress or eliminate T- or NK-cell activity for about 24 hours to about 7 weeks after administration of allo-HCT. In some embodiments, daily administration of compositions herein having at least one JAK inhibitor for about 1 day to about 28 days (e.g., about 1, 2, 3, 4, 5, 6, 7, 8 up to about 28 days) after allo-HCT can deplete CD3+ T cell production in the treated subject. In some embodiments, daily administration of compositions herein having at least one JAK inhibitor for about 1 to about 7 days (e.g., about 1, 2, 3, 4, 5, 6, 7 days) after allo-HCT can deplete CD4+ T cell production in the treated subject. In other embodiments, daily administration of compositions herein having at least one JAK inhibitor for about 1 to about 7 days (e.g., about 1, 2, 3, 4, 5, 6, or about 7 days) after allo-HCT can deplete CD8+ T cell production in the treated subject.

In some embodiments, transient, long-term, intermittent immunosuppressive treatment methods can be included in compositions and methods disclosed herein to reduce graph rejection or improve transplantation tolerance in a subject in conjunction with compositions and methods disclosed herein. Examples of immunosuppressive treatment regimens can include, but are not limited to, calcineurin inhibitors (e.g., tacrolimus, cyclosporine, pimecrolimus), antiproliferative agents (e.g., mycophenolate mofetil, mycophenolate sodium, azathioprine), mTOR inhibitors (e.g., sirolimus, everolimus), steroids (e.g., corticosteroids, prednisone), depleting antibodies (e.g., antithymocyte globulin, alemtuzumab, rituximab) non-depleting antibodies (e.g., basiliximab, daclizumab), belatacept, and the like. In some embodiments, these combination compositions and/or methods can further include administering an immunosuppressant to the subject depending on need for improving transplantation outcomes. In other embodiments, the combination composition or individual compositions can be administered to a subject before, during and/or after allograft transplantation or implantation.

In certain embodiments, methods disclosed herein can further include irradiation of the subject before, during or after HCT and administration of one or more JAK inhibitor. In some embodiments, methods can further include administering total body irradiation (TBI) to a subject. In certain embodiments, TBI can be either myeloablative (MA) dosing or reduced intensity conditioning (RIC) dosing. In some embodiments, TBI can be RIC dosing. In some embodiments, TBI can be at a dose between about 1 gray (Gy) to about 18 Gy (e.g., about 1, 2, 3, 4, 5, 6, 7, 8, 9, 10, 11, 12, 13, 13.2, 14 Gy). In some embodiments, TBI can be at a dose of about 2 Gy to about 5 Gy.

In some embodiments, methods herein can include administering at least one JAK inhibitor about 1 month, about 2 weeks, about 7 days, about 5 days, about 3 days or about 1 day or hours before HCT (e.g. allo-HCT) infusion. In certain embodiments, methods disclosed herein can include administering at least one JAK inhibitor about 28 days to about 1 day (e.g., about 28, 27, 26, 25, 24, 23, 22, 21, 20, 19, 18, 17, 16, 15, 14, 13, 12, 11, 10, 9, 8, 7, 6, 5, 4, 3, 2, 1 days) before HCT infusion. In some embodiments, methods disclosed herein can include administering at least one JAK inhibitor at least about 3 days after administering allo-HCT. In other embodiments, methods herein can include administering at least one JAK inhibitor about 14 days to about 1 day (e.g., about 14, 13, 12, 11, 10, 9, 8, 7, 6, 5, 4, 3, 2, 1 days) after administering allo-HCT.

In some embodiments, methods disclosed herein can include administering at least one JAK inhibitor at a dose between about 0.05 mg/kg/day to about 300 mg/kg/day (e.g., about 0.05, 0.1, 0.5, 1, 5, 10, 15, 20, 25, 30, 40, 50, 75, 100, 125, 150, 175, 200, 225, 250, 275, 300 mg/kg/day). Appropriate amounts or doses can be determined by those skilled in the art following routine practice. For example, different doses can be given to a subject and the serum level of the compound can be monitored at various time point after the administration to determine the suitable dose that would lead to the target serum level of the compound. In other embodiments, different doses can be given to a subject depending on the subject's age, gender, health status, disease/condition severity, genetic predisposition to treatment, and the like.

In some embodiments, methods disclosed herein can include administration of a JAK inhibitor or a pharmaceutical composition disclosed herein to a subject by various routes of administration, depending upon the dosage, the condition being treated, as well as the purpose for which it is being used. In accordance other embodiments, a JAK inhibitor or a pharmaceutical composition herein can be injected intravenously (intravenous, IV), by renal infusion or other or by oral administration (e.g., in tablet form), optionally after meals. In accordance with certain embodiments herein, a subject (e.g., a human patient or other mammal) can be treated by orally administering therapeutically effective amounts of a JAK inhibitor or a pharmaceutical composition thereof at a concentration of about 0.05 mg/kg/day to about 300 mg/kg/day. In accordance with certain embodiments, a subject (e.g., a human patient) can be treated by intravenously administering therapeutically effective amounts of a JAK inhibitor or a pharmaceutical composition thereof having a concentration of about 0.05 mg/kg/day to about 300 mg/kg/day. In accordance with these embodiments, these compositions can be administered before, during or after HCT and/or solid organ transplantation.

In some embodiments, methods disclosed herein can include administering a JAK inhibitor or a pharmaceutical composition disclosed herein to a subject at least once a day, at least twice a day, at least three times a day. In other embodiments, methods disclosed herein can include administration of a JAK inhibitor or a pharmaceutical composition disclosed herein to a subject every day, every other day, once a week, once every two weeks, once every three weeks, once a month, or once a year. In certain embodiments, a JAK inhibitor or a pharmaceutical composition containing one or more JAK inhibitor disclosed herein can be repeatedly administered to a subject as often as deemed necessary and effective by a health professional. In some embodiments, a pre-determined dosing and schedule can vary from patient to patient but can be determined by one or skill in the art for a particular patient such as a health professional or by measuring indicators disclosed herein. In certain embodiments, dosing schedules can be determined by assessing transplant or implant success or stability in the subject where additional treatments of JAK inhibitors can be administered when a subject is experiencing signs of rejection or early indicators of transplant rejection. One of skill in the art understands the early signs of rejection.

In certain embodiments disclosed herein mixed donor:recipient (D:R) chimeras can be generated, such that elements of both the donor and the recipient hematopoietic and immune systems are present in the transplant recipient. In accordance with these embodiments, tolerance to transplanted donor organs can be achieved after HCTs while dependency on toxic immunosuppressive drugs to prevent organ rejection is reduced or eliminated (e.g., by using JAK inhibitors at the time of transplantation to induce donor cell engraftment and/or mixed D:R chimerism, etc.). By these examples, two immune systems (e.g., the patient and the donor (allogenic donor)) can be introduced and tolerated. This is an important discovery because in the solid organ transplantation it has been shown that mixed donor:recipient chimerism is associated with tolerance to the transplanted organ and can permit dramatically reduced or eliminated need for immune suppression after organ transplantation.

In some embodiments, kits are contemplated. In certain embodiments, a kit can include one or more JAK inhibitor or pharmaceutical composition containing one or more JAK inhibitor disclosed herein and at least one container. In other embodiments, kits can further include one or more delivery devices. In yet other embodiments, kits can include devices for obtaining donor cells for allo-HCT and further include at least one JAK inhibitor or a pharmaceutical composition disclosed herein. In other embodiments, kits can include compositions of harvested HCTs and one or more JAK inhibitors. In other embodiments, JAK inhibitors can be included in kits that accompany solid organs to be transplanted with or without HCTs.

In other embodiments, kits disclosed herein can additionally include instructions for using one or more JAK inhibitor or pharmaceutical compositions including one or more JAK inhibitor disclosed herein for use in any of the methods described herein. In accordance with certain embodiments, the instructions can include a description for administering the one or more JAK inhibitor or pharmaceutical compositions disclosed herein in conjunction with allo-HCT and/or solid organ transplantation to a subject to achieve the intended effect in the subject.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of embodiments of the inventions.

### Example 1

In one exemplary method, mouse models of human allogeneic bone marrow transplant events were prepared. In brief, major histocompatibility complex (MHC)-mismatched bone marrow transplants (BMTs, HCT)—which model human allogeneic BMTs—were performed in mice. Mice used in this exemplary method were the C57BL/6 mouse strain (which express the MHC marker H2K^{b}) and the Balb/cJ strain (which express the MHC marker H2K^{d}). Both mouse strains were maintained by homozygous breeding according to established vivarium practices. Mice approximately 6-10 weeks of age were used for both recipients and donors. Donors and recipients were matched in gender (male recipient mice receive cells from male donors while female recipients receive cells from female donors).

In another exemplary method, BMT was performed by isolating bone marrow cells from the bones of euthanized donor mice using a method similar to previous methods (Amend et al., J Vis Exp. 2016 Apr. 14; (110):53936). In brief, bone marrow cells were resuspended in room temperature (25±3° C.) red blood cell (RBC) lysis buffer which has been diluted to 1× concentration using ddH2O in 50 mL tubes. RBCs were lysed according to manufacturer instructions. The resulting cell suspension was centrifuged at 1500-1700 rpm for 5 minutes. The supernatant was aspirated by vacuum aspirator and the cell pellet was resuspended using cold phosphate-buffered saline (PBS) prior to filtering through a 70 μm cell strainer. An aliquot of the cell suspension was removed and mixed 1:1 with Trypan Blue. Viable cells (which excluded Trypan Blue) were counted using a hematocytometer. The cell suspension was centrifuged at 1500-1700 rpm for 5 minutes prior to resuspension in appropriate volume of PBS to achieve the desired cell concentration (10-20 million cells per 100-200 μL, the volume of cells delivered to each recipient mouse). The bone marrow cells were administered to mice using 29 G syringes and injected via lateral tail vein. Peripheral blood was then collected from transplant recipients at 4 and 6 weeks and at indicated timepoints thereafter after BMT to assess for donor cell engraftment. 50-100 μL of blood was obtained from each mouse by tail vein nicking. Blood was subjected to RBC lysis as described herein. Following RBC lysis, blocking of FcX receptors was performed using Biolegend FcX Plus per manufacturer instructions and then cells were stained with fluorescently labelled antibodies (Biolegend or BD Biosciences) against the following cell markers: CD45 (hematopoietic cells), Ter119 (RBCs), H2K^{b} (C57BL/6), H2K^{d} (Balb/cJ), CD3 (T cells), NKp46 (NK cells), CD19 (B cells) and CD11b (monocytes) in addition to staining with a cell viability dye (Tonbo). Following a 30-minute staining incubation, cells were washed twice by addition of PBS followed by centrifugation and removal of supernatant by manual pouring. Stained cells were resuspended in PBS and evaluated via Fluorescent-Activated Cell Sorting (FACS) on a Cytek Aurora cytometer. FACS data was analyzed using FloJo software. Using forward versus side scatter, the cell population of interest was gated, and additional sub-gating was performed to exclude doublets and dead cells (which stain positively with viability dye). Cells which were negative for Ter119 (RBCs) and positive for CD45 (hematopoietic cells) were further analyzed. Cells which stained positively for donor versus recipient cell markers (H2K^{b} or H2K^{d}) were reported while additional cell types (T cells, B cells, NK cells and monocytes) were also analyzed to ensure complete donor cell engraftment in all immune cell types.

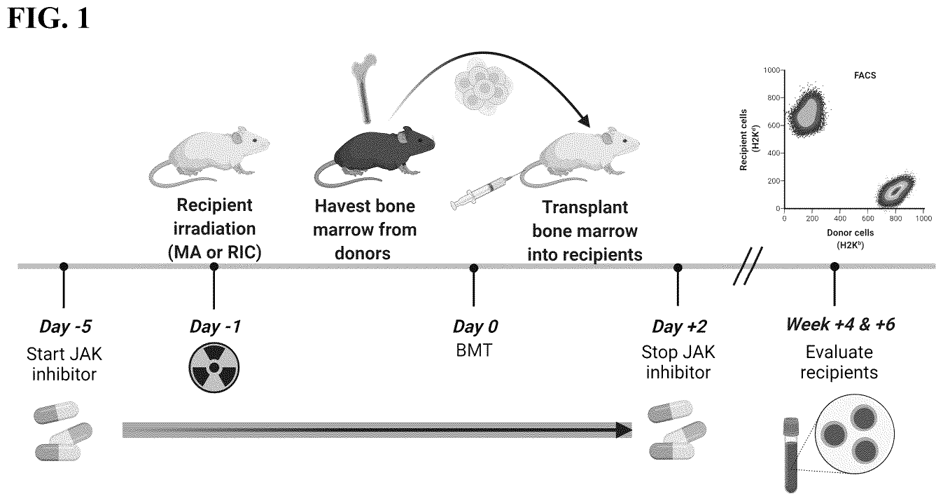

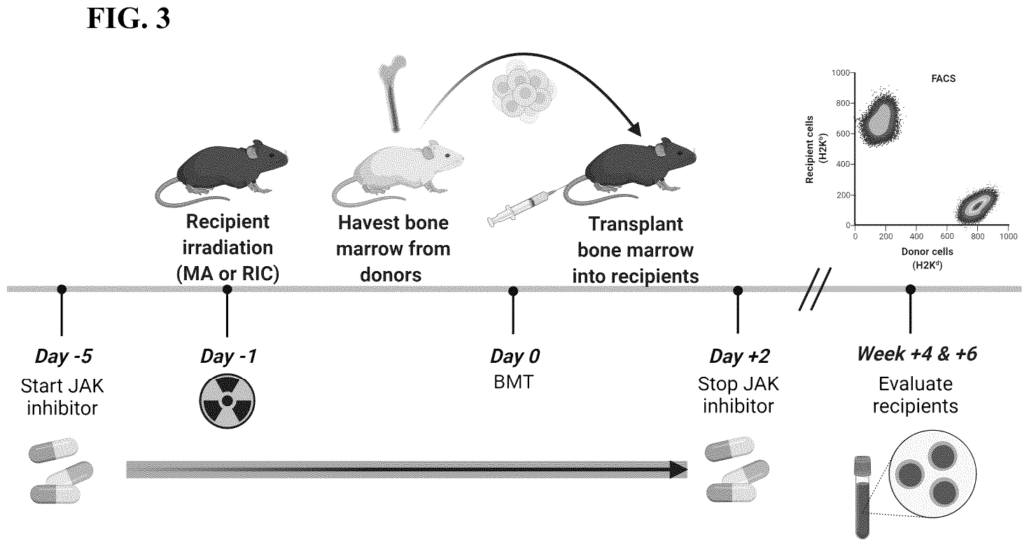

In another exemplary method, BMTs were performed as outlined in FIG. 1 and FIG. 3. In brief, recipient mice were assigned to the control or experimental group. Control mice received conditioning with total body irradiation (TBI) at either myeloablative (MA) dosing or reduced intensity conditioning (RIC) dosing. The MA dose was dependent on the recipient strain (reduced for Balb/cJ given increased radiation sensitivity) and the RIC dose was set at 50% of the strain-specific MA dose. Control mice which received MA TBI were designated positive controls as they were expected to demonstrate successful engraftment of donor cells 100% of the time. Control mice which received only RIC TBI were designated negative controls as they are expected to reject donor cells 100% of the time. All mice were switched from standard mouse chow to Uniprim medicated chow (Envigo) on the day of irradiation to minimize infectious complications.

In yet another exemplary method, mice in the experimental group received reduced TBI dosing in addition to a JAK inhibitor. The JAK inhibitor was administered via oral gavage twice daily beginning 5 days before BMT (Day −5) and ending on Day +2 after transplant for a total of 8 days. (See FIG. 1 and FIG. 3) The dose of JAK inhibitor given to each individual mouse was calculated based on the weight of the mouse at the start of treatment. The JAK inhibitors Ruxolitinib phosphate was purchased (e.g., LC Laboratories) and prepared as stock solutions by dissolving in DMSO according to manufacturer reported solubility at 200 mg/ml. This stock solution was aliquoted into Eppendorf tubes and kept at −80° C. until use. Prior to administration, the stock solutions were further diluted with ddH2O to an appropriate concentration dependent on the dose delivered, such that volume delivered to each mouse was 50-100 μL per gavage.

### Example 2

In another method, BMTs were performed and assessed as outlined in FIG. 1. In this exemplary method, BMTs were performed in mice as described in Example 1 wherein Balb/cJ mice (which express H2K^{d}) were used as transplant recipients while C57BL/6 mice (which express H2K^{b}) served as stem cell donors. Positive controls received a MA TBI dose of 8 Gy while negative controls and experimental mice received a RIC TBI dose of 4 Gy. TBI was delivered via a ¹³⁷Cs irradiator in 2 equal fractions approximately 4 hours apart on Day −1 relative to BMT (which occurs on Day 0).

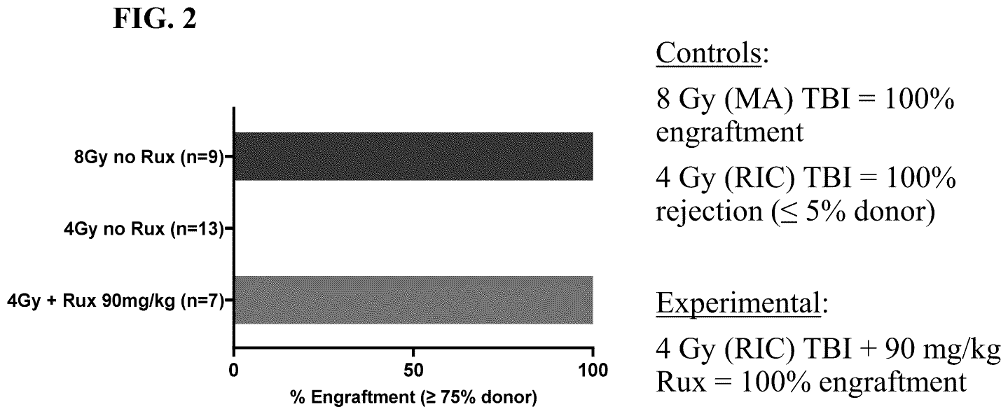

FIG. 2 illustrates the results of BMTs ((HCT) engraftment or rejection) using Balb/cJ recipients and C57BL/6 donors at 4 weeks post-BMT after TBI RIC (Gy), with or without ruxolitinib (a JAK 1/2 inhibitor) treatment at the indicated treatment times. 100% of mice who received myeloablative (MA) TBI (positive controls) demonstrated donor cell engraftment and 100% of mice who received reduced-intensity (RIC) TBI without a JAK inhibitor (negative controls) demonstrated donor cell rejection in both recipient/donor strain combinations used. For initial BMTs using Balb/cJ recipients and C57BL/6 donors, mice that received a reduced 4 Gy RIC in addition to 90 mg/kg ruxolitinib (Rux) from Day −5 through Day +2 demonstrated 100% donor cell engraftment (n=7). Ruxolitinib administered five days before BMT (D-5) thru two days after BMT (D+2) for a total of 8 days of treatment was determined to be highly successful dosing regimen because it provided adequate treatment before BMT that was greater than ablation of recipient lymphocytes (including T cells) while also providing continuing treatment after BMT which prevented premature rebound of recipient lymphocytes (which can cause transplant rejection). It is contemplated that JAK inhibitor treatment regimens can include a regimen beginning about 6 months or about 5 months or about 4 months or less before implantation (HCT) to about 1 day, to about 3 days, to about a week, to about 10 days, to about 2 weeks, to about 1 month, to about 2 months to up to about 6 months after implantation. In certain exemplary methods, JAK inhibitors can be given to a subject undergoing HCT once daily, twice daily, every other day, every few days or as needed to the subject. In some methods, JAK inhibitors can be provided to a subject undergoing HCT 2 times daily.

### Example 3

In another exemplary method, BMTs were performed and assessed as outlined in FIG. 3. In this exemplary method, BMTs were performed in mice as described in Example 1 wherein C57BL/6 mice (which express H2K^{b}) were the transplant recipients while Balb/cJ mice (which express H2K^{d}) served as stem cell donors. Positive controls received a MA TBI dose of 13 Gy while negative controls and experimental mice received a RIC TBI dose of 6.5 Gy. Additionally, weight-based doses of the JAK inhibitor Ruxolitinib were given using the same dosing regimens described in Example 2 above.

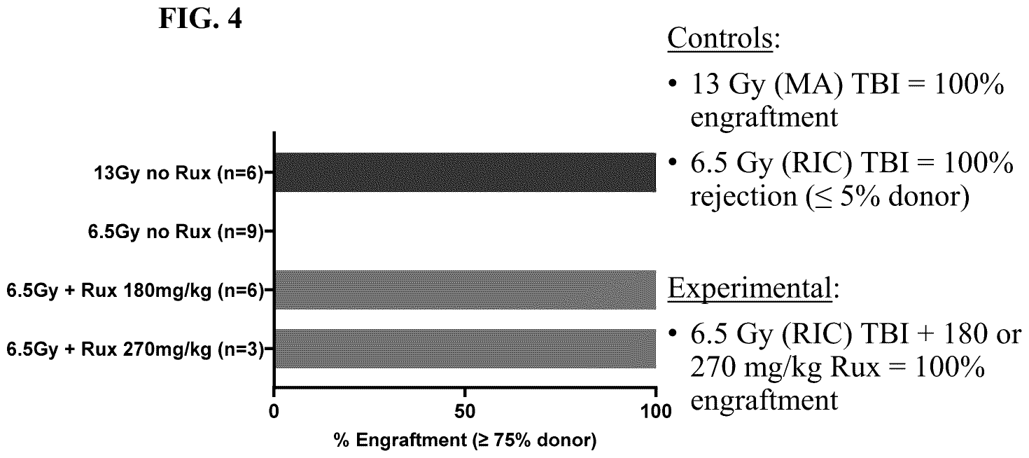

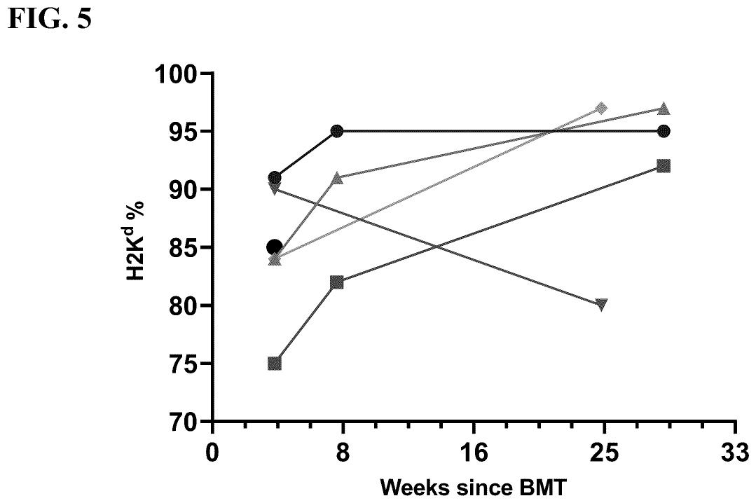

FIG. 4 demonstrates the results of BMTs ((HCT) engraftment or rejection) using C57BL/6 recipients Balb/cJ donors at 4 weeks post-BMT after TBI RIC (Gy) with or without ruxolitinib (a JAK 1/2 inhibitor) treatment at the indicated treatment times. 100% of mice who received myeloablative (MA) TBI (positive controls) demonstrated donor cell engraftment and 100% of mice who received reduced-intensity (RIC) TBI without a JAK inhibitor (negative controls) demonstrated donor cell rejection in both recipient/donor strain combinations used. For BMTs using C57BL/6 recipients and Balb/cJ donors, mice that received a reduced 6.5 Gy RIC in addition to 180 mg/kg (n=6) or 270 mg/kg (n=3) ruxolitinib (Rux) from Day −5 through Day +2 demonstrated 100% donor cell engraftment. Ruxolitinib administered five days before BMT (D-5) thru two days after BMT (D+2) for a total of 8 days of treatment was determined to be highly successful dosing regimen because it provided adequate treatment before BMT that was greater than ablation of recipient lymphocytes (including T cells) while also providing continuing treatment after BMT which prevented premature rebound of recipient lymphocytes (which can cause transplant rejection). It is contemplated that JAK inhibitor treatment regimens can include a regimen beginning about 6 months or about 5 months or about 4 months or less before implantation (HCT) to about 1 day, to about 3 days, to about a week, to about 10 days, to about 2 weeks, to about 1 month, to about 2 months to up to about 6 months after implantation. In certain exemplary methods, JAK inhibitors can be given to a subject undergoing HCT once daily, twice daily, every other day, every few days or as needed to the subject. In some methods, JAK inhibitors can be provided to a subject undergoing HCT 2 times daily.

### Example 4

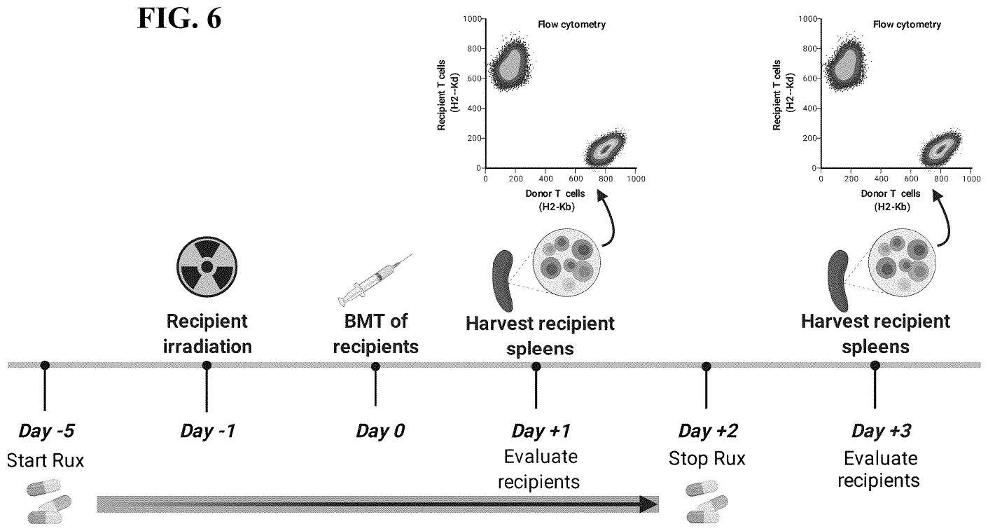

In another exemplary method, BMTs were performed and assessed to determine the effects of JAK inhibitors on recipient compared to donor NK and T cells as outlined in FIG. 6. In this exemplary method, BMTs were performed in mice as described in Example 1 wherein C57BL/6 mice were the transplant donors while Balb/cJ mice served as stem cell recipients. Recipient mice were treated with Ruxolitinib and given RIC TBI followed by BMT as previously described in the examples herein. On Day +1 and Day +3 post-BMT, a portion of the recipient mice were euthanized, and their spleens were harvested. The spleen cells were prepared into a single cell suspension, subjected to RBC lysis, stained (using previously referenced markers in addition to T-cell subset markers, CD4 and CD8) and analyzed by FACS as described in the examples herein.

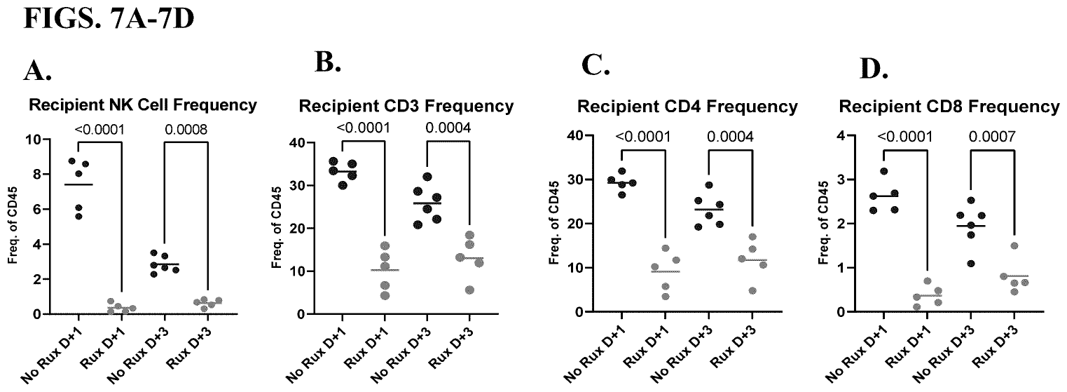

FIGS. 7A-7D illustrate effects of ruxolitinib on recipient T cells. In this example, mouse spleen cells were analyzed, and it was found that treatment of mice with ruxolitinib led to a statistically significant decrease in total recipient NK+ cells (FIG. 7A); CD3+ T cells (FIG. 7B) which was also reflected in decreased CD4+ (FIG. 7C) and CD8+ (FIG. 7D) T cell subsets. Notably, ruxolitinib had no significant effect on donor T cells (data not shown).

These exemplary methods demonstrated that JAK inhibitors added to a reduced intensity conditioning regimen led to successful engraftment in MHC-mismatched allogeneic BMTs in an acceptable mouse model. As one possible mechanism, JAK inhibitors were likely effective in promoting engraftment by selectively suppressing recipient lymphocytes, including T cells, which would otherwise cause rejection. The results of the exemplary methods herein demonstrate that administration of a JAK inhibitor to a subject undergoing allogenic BMT can reduce rates of transplant-associated morbidity and mortality and can support expanded indications for allogeneic BMT to other serious chronic conditions for which BMT is currently too much of a risk, but no other curative options are available to provide improved engraftment with reduced implantation rejection.

### Example 5

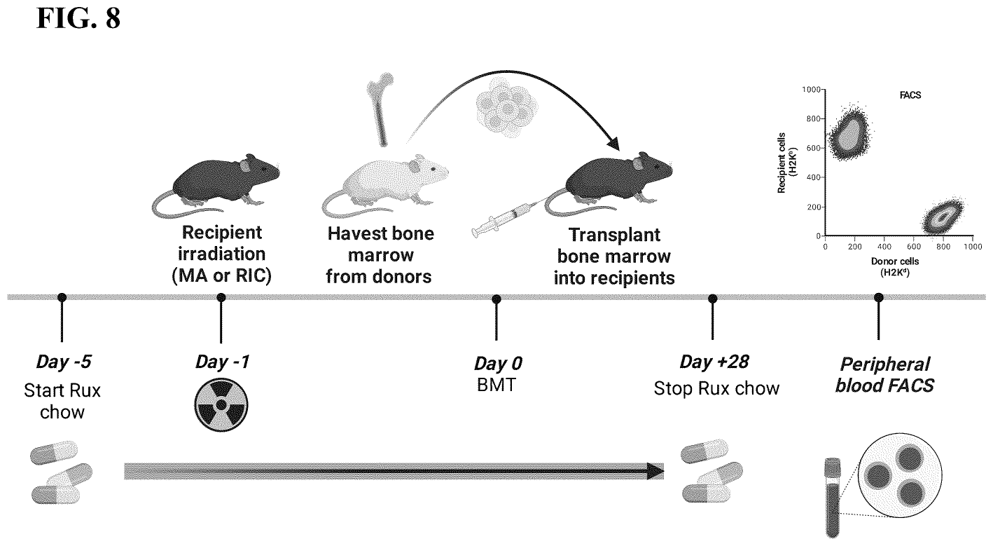

In yet another exemplary method, BMTs were performed and assessed as outlined in FIG. 8. In this exemplary method, BMTs were performed in mice as described in Example 1 wherein C57BL/6 mice (which express H2K^{b}) were the transplant recipients while Balb/cJ mice (which express H2K^{d}) served as stem cell donors. Positive controls received a MA TBI dose of 13 Gy, negative controls received a RIC TBI dose of 6.5 Gy and experimental mice received a RIC TBI dose of 6.5 Gy or a further de-escalated TBI dose of 4 Gy or 2 Gy in addition to JAK inhibitor. In this method, the JAK inhibitor Ruxolitinib was given to mice beginning 5 days before BMT (Day −5) and ending on Day +28 after transplant for a total of 34 days. In this method, Ruxolitinib was provided to mice in the form of Ruxolitinib-impregnated chow which was prepared at a concentration of 2 grams of Ruxolitinib per kg chow mix, which was provided to mice continuously in place of unmedicated mouse chow.

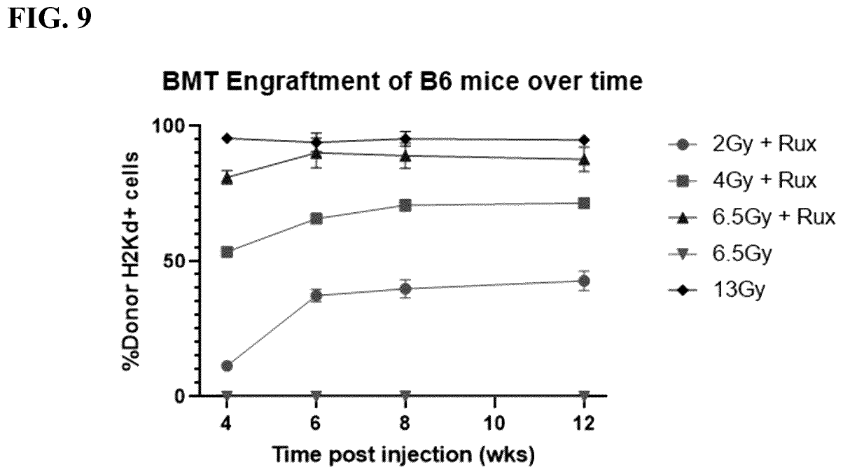

FIG. 9 demonstrates the degree of donor cell engraftment using C57BL/6 recipients Balb/cJ donors at 4 to 12 weeks post-BMT after indicated doses of TBI (Gy) with or without ruxolitinib (a JAK 1/2 inhibitor) treatment. Ruxolitinib administered five days before BMT (D−5) thru 28 days after BMT (D+28) for a total of 34 days enabled generation of stable donor:recipient mixed chimerism utilizing a very low dose of 2 or 4 Gy TBI. Utilization of low-dose TBI in addition a JAK inhibitor as part of allo-HCT could be utilized to induce stable mixed chimerism. This procedure would be beneficial for patients who have received or are going to receive a solid organ transplantation. The induction of stable mixed chimerism in such patients would likely enable a reduction or elimination in the patients need for lifelong immunosuppression. Induction of stable mixed chimerism in solid organ transplant recipients may also prevent “micro-rejection” episodes, thereby prolonging the viability of the engrafted organ.

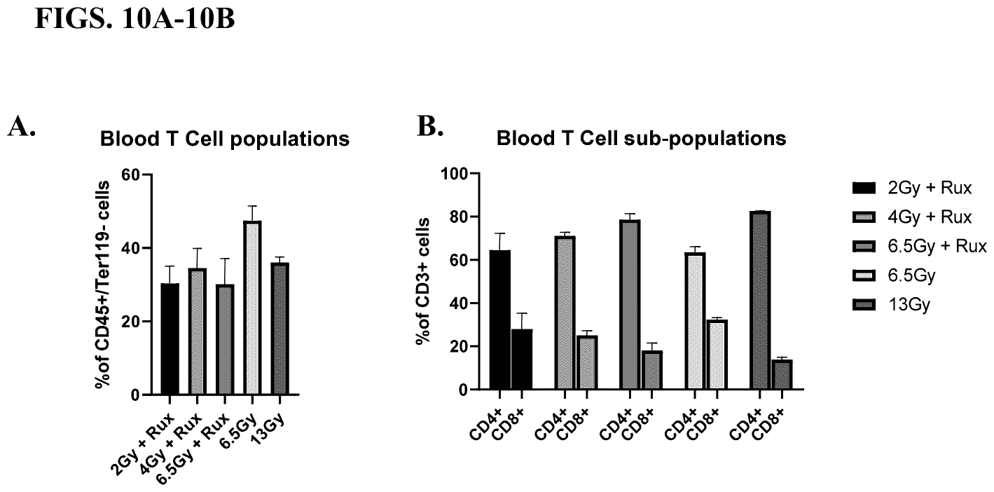

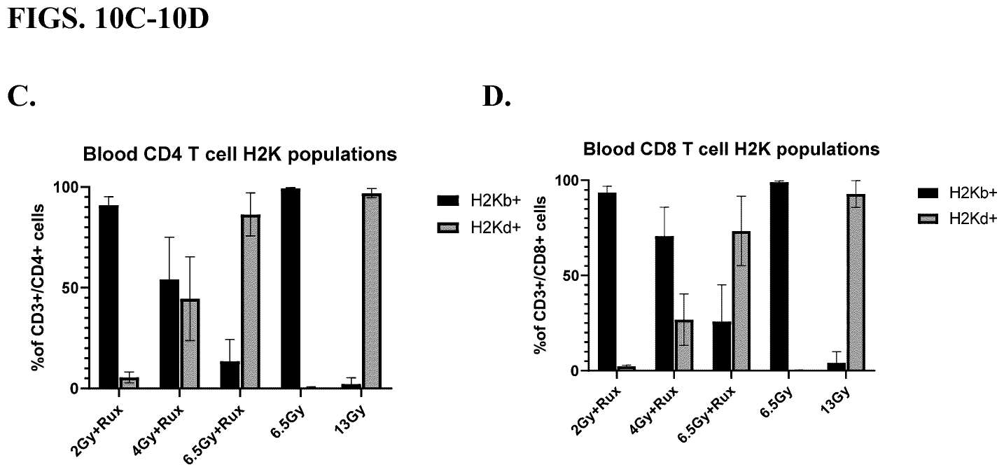

FIGS. 10A-10D illustrate the recipient T cells 12 weeks after BMT as illustrated in FIG. 8. In this example, mouse spleen cells were analyzed. (FIG. 10A) indicates the percentage of splenocytes that are T cells in the various conditions. (FIG. 10B) indicated CD4 and CD8 T cell percentages showing a ˜2:1 (CD4:CD8) ratio which is expected. Percentage donor CD4 T cells are shown in (FIG. 10C) and donor CD8 T cells (FIG. 10D) under indicated conditions.

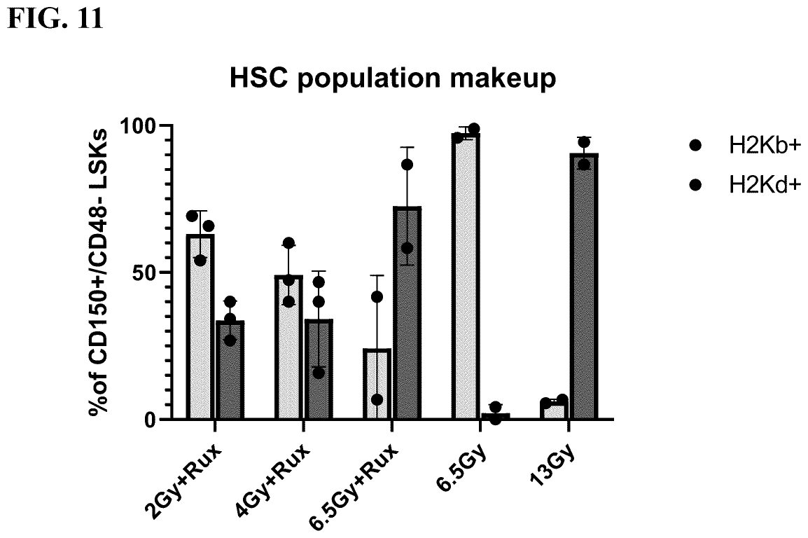

FIG. 11 illustrates examination of hematopoietic stem cells (HSCs) 12 weeks after allogeneic bone marrow transplantation showing engraftment of long-term engrafting stem cells (Lin−LSK+CD48−CD150+).

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of reducing allogeneic hematopoietic cell transplantation (allo-HCT) rejection in a subject having undergone, undergoing, or scheduled to undergo an allo-HCT comprising, administering to the subject a composition containing at least one Janus kinase (JAK) inhibitor to a subject and improving engraftment while reducing or eliminating the need for at least one of cytotoxic chemotherapy and irradiation of the subject.

2. The method according to claim 1, wherein the subject has or is suspected of developing a non-malignant or malignant condition; or has undergone or is undergoing solid organ or non-organ cellular transplantation.

3. The method according to claim 1, wherein the composition comprising the at least one JAK inhibitor comprises administering at least one of, before, during or after providing the allo-HCT to the subject.

4. The method according to claim 1, wherein the composition comprising the at least one JAK inhibitor comprises administering the at least one JAK inhibitor to the subject about 1 month before providing the allo-HCT to the subject.

5. The method according to claim 1 or 4, wherein the composition comprising the at least one JAK inhibitor is administered about 1 day to about 1 week after providing the allo-HCT to the subject.

6. The method according to any one of claims 1-5, wherein the composition comprising the at least one JAK inhibitor comprises a concentration of the at least one JAK inhibitor of about 0.05 mg/kg to about 300 mg/kg.

7. The method according to any one of claims 1-6, wherein the at least one JAK inhibitor comprises a JAK1 inhibitor, a JAK2 inhibitor, a JAK3 inhibitor, a JAK1/2 inhibitor, a JAK1/3 inhibitor, a JAK2/3 inhibitor, a JAK1/2/3 inhibitor, or a combination thereof.

8. The method according to any one of claims 1-7, wherein the at least one JAK inhibitor comprises ruxolitinib, tofacitinib, baricitinib, upadacitinib, fedratinib, peficitinib, decernotinib, filgotinib, solcitinib, itacitinib, SHR0302, abrocitinib, delgocitinib, cerdulatinib, gandotinib, lestaurtinib, momelotinib, pacritinib, deucravacitinib, cucurbitacin I, CHZ868, oclacitinib, XL019, AZD1480, BMS911543, ilginatinib, or a combination thereof.

9. The method according to claim 1, wherein the at least one JAK inhibitor comprises ruxolitinib.

10. The method according to any one of claims 1-9, further comprising administering at least one of total body irradiation (TBI) of about 1 Gy to about 16 Gy.

11. The method according to any one of claims 1-10, further comprising reducing or eliminating chemotherapeutic treatments of the subject.

12. The method according to any one of claims 1-11, wherein the subject is a human subject.

13. The method according to claim 1 or 2, wherein the non-malignant or malignant condition comprises bone marrow failure, an immune dysregulation syndrome, a hemoglobinopathy, an immunodeficiency, a metabolic disorder, ALL, AML, lymphoma, MDS, CML or a combination thereof.

14. The method according to claim 1 or 2, wherein the solid organ transplant comprises one or more of a kidney, heart, lung, liver, pancreas, gastrointestinal tract transplantation or other solid organ.

15. The method according to claim 1 or 2, wherein the non-organ cellular transplant comprises an islet cell transplantation.

16. The method according to any one of claims 1-13, wherein the non-malignant condition comprises familial hemophagocytic lymphohistiocytosis (FHL), Wiskott-Aldrich syndrome (WAS), T-cell deficiency, metachromatic leukodystrophy (MLD), Lagerhans cell histiocytocis, erythropoietic protoporphyria, adenosine deaminase deficiency (ADA), Maroteaux-Lamy syndrome (MPS VI), Fanconi syndrome, thrombocytopenia, aspartylglucosaminuria (AGU), adrenoleukodystrophy (ALD), chronic granulomatous disease (CGD), thalassemia major, Hurler syndrome, Kostmann syndrome, severe combined immunodeficiency (SCID), sickle cell anemia, paroxysmal nocturnal hematoblobunimeuria (PNH), or a combination thereof.

17. The method according to any one of claims 1-16, wherein the subject does not have myelofibrosis or rheumatoid arthritis.

18. A composition comprising: HCTs; and at least one Janus kinase (JAK) inhibitor.

19. The composition according to claim 18, wherein the HCTs comprise allo-HCTs.

20. The composition according to claim 18 or 19, wherein the at least one JAK inhibitor comprises ruxolitinib, tofacitinib, baricitinib, upadacitinib, fedratinib, peficitinib, decernotinib, filgotinib, solcitinib, itacitinib, SHR0302, abrocitinib, delgocitinib, cerdulatinib, gandotinib, lestaurtinib, momelotinib, pacritinib, deucravacitinib, cucurbitacin I, CHZ868, oclacitinib, XL019, AZD1480, BMS911543, ilginatinib, or a combination thereof.

21. The composition according to any one of claims 18-20, wherein the composition is formulated for intravenous administration.

21. The composition according to any one of claims 18-20, wherein the composition is formulated for oral administration.

22. The composition according to any one of claims 18-21, wherein the composition comprises about 0.05 mg to about 300 mg of the at least one JAK inhibitor.

23. A method of treating or reducing the onset of a non-malignant condition in a subject comprising administering a composition according to any one of claims 18-22 to the subject, wherein the subject has or is suspected of developing a non-malignant disease.

24. A kit comprising one or more compositions according to any one of claims 18-22, and at least one container.

25. The kit according to claim 24, further comprising a device for delivering the one or more compositions to a subject.
